(19) United States
(12) Patent Application Publication
(10) Pub. No.: US 2005/0196835 A1
(43) Pub. Date: Sep. 8, 2005

(54) Cold-active beta galactosidase, the process for its preparation and the use thereof

(75) Inventors: Charles Gerday, Esneux (BE); Anne Hoyoux, Tilff (BE); Jean-Marie Francois, Soheit-Tinlot (BE); Phillip Dubois, Liege (BE); Etienne Baise, Binche (Buvrinnes) (BE); Isabell Jennes, Charneux (BE); Sabine Genicot, Roscoff (FR)

Correspondence Address: VENABLE LLP, P.O. BOX 34385, WASHINGTON, DC, 20045-9998, US

(73) Assignee: University de Liege, Liege (BE)

(21) Appl. No.: 10/803,055

(22) Filed: Mar. 18, 2004

Related U.S. Application Data
(62) Division of application No. 09/501,136, filed on Feb. 9, 2000, now Pat. No. 6,727,084.
(60) Provisional application No. 60/143,114, filed on Jul. 9, 1999.

(51) Int. Cl. C07H 21/04; C12N 9/38; C12N 1/21; C12N 15/74

(52) U.S. Cl. 435/69.1; 435/207; 435/252.3; 435/471; 536/23.2

(57) ABSTRACT

The present invention relates, for example, to an isolated DNA comprising a sequence which encodes a cold-active beta galatosidase that is specific for lactose and has a stable enzymatic activity at a temperature below 8° C. In embodiments of the invention, the DNA is isolated from the psychrophilic bacterium Pseudoalteromonas haloplanktis.

### CROSS-REFERENCE TO RELATED APPLICATION

This application is a divisional of co-pending U.S. application Ser. No. 09/501,136, filed Feb. 9, 2000 and claims the benefit of U.S. provisional application Ser. No. 60/143,114, filed Jul. 9, 1999, each of which is hereby incorporated by reference in its entirety.

### FIELD OF THE INVENTION

The present invention relates to a purified β-galactosidase specific for lactose.

β-galactosidase catalyzes the hydrolysis of lactose disaccharide into its constituent monosaccharides, glucose and galactose.

This enzyme is widely distributed in numerous micro-organisms, βlant and animal tissues.

### DESCRIPTION OF THE RELATED ART

The ability of β-galactosidase to hydrolyse lactose into galactose is applied in food industry, particularly in the field of dairy products because of the nutritional (lactose intolerance), technological (crystallisation) and environmental (pollution) problems associated with lactose (Triveni P. S., 1975, CRC Critical Reviews in Food Technology 325-354). The added value gained by the hydrolysis of lactose, to its constituent monosaccharides glucose and galactose, lies in the increased usefulness of hydrolysed lactose as a food carbohydrate. Lactose itself has limited use in this respect because of its relatively low sweetness, solubility and digestibility, but the hydrolysis products of lactose, i.e. glucose and galactose, are superior in all of these respects. Increased sweetness and solubility improve the technical usefulness of whey products while the increased digestibility of hydrolysed lactose also offers the opportunity of supplying milk solids to populations which have hitherto been unable to consume milk products because of their inability to hydrolyse lactose in the digestive tract.

The β-galactosidase can be applied to the production of low-lactose milk and in the production of galactose or glucose from lactose contained in milk serum which is formed in large amount in the process of producing cheese.

The major applications for lactose hydrolysis are listed below.
a) Liquid milk. Lactose hydrolysis in liquid milk improves digestibility for lactose intolerant consumers. In flavoured milks, lactose hydrolysis increases sweetness and enhances flavours.
b) Milk powders. Lactose hydrolysed milk powders for dietetic uses, especially for infants with temporary β-galactosidase deficiency.
c) Fermented milk products. In some cases, lactose hydrolysis in milk used for the manufacture of cheese and yoghurt can increase the rate of acid development and thus reduce processing time.
d) Concentrated milk products. Lactose hydrolysis in concentrated milk products (e.g. sweetened condensed milk, ice cream) prevents crystallisation of lactose.
e) Whey for animal feed. Lactose hydrolysis in whey enables more whey solids to be fed to pigs and cattle and also prevents crystallisation in whey concentrate.
f) Whey. Lactose hydrolysed whey is concentrated to produce a syrup containing 70-75 per cent solids. This syrup provides a source of functional whey protein and sweet carbohydrate and is used as a food ingredient in ice cream, bakery and confectionery products.

The conventional approach in food processing is to carry out the hydrolysis of lactose at 40° C. during approximately four hours. (T. Godfrey and J. Reichelt in : “Industrial Enzymology: the application of enzymes in industry”; The Nature Press, Mac Millan Publishers Ltd, GB, 1983). However, milk or lactose solution as a raw material is a preferable nutrition source for bacteria. As the result, the putrefaction owing to the saprophyte contamination during the treatment is a serious problem in the food production. Thus, the fact is that the conventional β-galactosidase is not put into practical use.

Attempts to solve these problems consisted in using thermophilic enzymes as described in U.S. Pat. No. 4,237,230 and U.S. Pat. No. 4,007,283 but a problem of high energetic cost still remains.

On another hand, cold-adapted β-galactosidases have been studied (Trimbur D. E. and al., 1994, Appl. Environ. Microbiol. 60:4544-4552; Rahim K. A. A. and Leb B. H., 1991, Biotechnol and Appl. Biochem., 13, 246-256).

However, these β-galactosidases generally known in the prior art, when used for food processing, all have one or more disadvantages such as low enzyme activity and low stability at a temperature below 20° C., narrow range of optimum pH and the inhibition of enzymatic action by a reaction product, such galactose or others products particularly calcium.

### SUMMARY OF THE INVENTION

The object of the present invention is to hydrolyse lactose by using a β-galactosidase, which could overcome the above-mentioned drawbacks which are usually associated to this process, while advantageously avoiding contamination problems during the hydrolysis process and lowering the energy consumption.

This problem is solved according to the present invention by a purified cold-active β-galactosidase, specific for lactose, having a stable enzymatic activity at temperatures up to below 8° C., preferably up to below 6° C., and specifically at 4° C., which corresponds to refrigerating conservation temperature for dairy products. This enzyme of the invention is consequently able to hydrolyse lactose in dairy products and stable enzymatic activity at temperatures up to below 8° C., preferably up to below 6° C., and specifically at 4° C., which corresponds to refrigerating conservation temperature for dairy products. This enzyme of the invention is consequently able to hydrolyse lactose in dairy products and milk processing at such a low temperature that saprophytes are hindered to proliferate. The hydrolysis of lactose can be carried out in these refrigeration conditions with no need of a particular treatment to the dairy product concerned.

According to the invention, an enzymatic activity is considered as stable when, in the concerned conditions, the enzyme is capable of lasting long enough to obtain the desired effect, for example, the hydrolysis of a substrate.

According to an embodiment of the invention, the cold-active β-galactosidase has a stable enzymatic activity between 0 and 50° C.

Advantageously, the cold-active β-galactosidase according to the invention has a stable enzymatic activity at a pH range from 6 to 10, preferably from 6 to 8.

Preferably, the cold-active β-galactosidase according to the invention has a stable enzymatic activity in presence of calcium and/or galactose, meaning that the activity of this enzyme is neither inhibited by its reaction product nor by products being present in milk. This property allows to use efficiently this enzyme in milk treatment.

Such a cold-adapted β-galactosidase according to the invention attains the level of practical application, having simultaneously the following properties:
(1) Having a sufficient stability in the neighbourhood of 0 to 10° C.
(2) Having a sufficient enzymatic activity at a pH range from 6 to 10
(3) Having an enzymatic activity non inhibited by reaction products or other products substantially present in milk, such calcium.

According to an advantageous embodiment of the invention, the enzyme can be inactivated at a pasteurisation temperature. This property of the enzyme according to the present invention allows to apply the β-galactosidase according to the invention and to stop the enzymatic reaction of lactose hydrolysis without any additional step during a current milk treatment.

Another object of the present invention is a strain of an isolated psychrophilic bacterium capable of producing a cold-active β-galactosidase according to the present invention. A preferable strain is Pseudoalteromonas haloplanktis deposited on the 4^{th} of Nov., 1999, under the Budapest Treaty at the Belgian Coordinated Collections of Microorganisms (BCCM™), Laboratorium voor Microbiologie—Bacteriënverzameling (BCCM™/LMG), Universiteit Gent, K. L. Ledeganckstraat 35, 9000 Gent, Belgium, with the Accession N° LMG P-19143 and variants and mutants derived therefrom.

To purify a cold-active β-galactosidase according to the invention, a bacterium living in the Antarctic area was isolated and characterised in order to study how its enzymes, and particularly, the β-galactosidase was adapted to cold. These studies led to the purification of the β-galactosidase, meaning that this protein was obtained substantially free of other proteins as determined by Sodium Dodecyl Sulphate Polyacrylamide gel Electrophoresis (SDS-PAGE) using protein purification steps known in the art.

Micro-organisms can be divided in categories depending on the temperature at which they can proliferate. The widely accepted definition by Morita (Psychrophilic bacteria. Bacteriol. Rev. 39: 144-167; 1975.) proposes that psychrophiles include organisms having optimum growth temperatures <15° C. and upper cardinal temperatures around 20° C., although they are able to multiply and to carry out all their biochemical functions near the normal freezing point of water. The mesophilic bacteria proliferate at an average temperature range between 25 and around 40° C. Thermophilic micro-organisms proliferate at a temperature above 50° C. and hyperthermophilic micro-organisms grow at temperatures above 80° C.

As a general rule, micro-organisms which are pathogenic for human and animals are mesophilic, so it is interesting to carry out industrial food processing at low temperatures to avoid the possible proliferation of such pathogens.

It is still an object of the present invention to provide a DNA sequence comprising a gene which encodes a polypeptide having the biological activity of the cold-active β-galactosidase according to the invention. A preferable DNA sequence is shown in SEQ ID NO: 1 and a polypeptide having an amino acid sequence as shown in SEQ ID NO: 2 is preferable.

Another object of the invention is a recombinant plasmid suited for transformation of a host, capable of directing the expression of a DNA sequence according to the invention in such a manner that the host expresses said polypeptide having the biological activity of the cold-active β-galactosidase in recoverable form. According to the invention another object is the so transformed host.

A variety of host-expression systems may be conceived to express the cold-active ,β-galactosidase coding sequence, for example bacteria, yeast, insect cells, plant cells, mammalian cells, etc.

Particularly, in yeast, a number of vectors containing constitutive or inducible promoters may be used. For a review see Grant and al., 1987, Expression and secretion vectors for yeast, in Methods in Enzymology, Eds. Wu & Grossman, 31987, Acad. Press, N.Y., Vol. 153, pp. 516-544.

It is also an object of the present invention to provide a process for purifying the cold-active β-galactosidase according to the invention from a psychrophilic bacterium as well as to provide a process for producing cold-active β-galactosidase according to the invention in a transformed host.

These and other objects of the present invention will be apparent from the following disclosure.

Other characteristics of the present invention are listed in the annexed claims.

### BRIEF DESCRIPTION OF THE DRAWINGS

FIG. 1a shows the growth of the strain Pseudoalteromonas haloplanktis LMG P-19143 at different temperatures.

FIG. 1b shows the cell viability and the cold-active β-galactosidase activity of the strain Pseudoalteromonas haloplanktis LMG P-19143 versus temperatures.

FIG. 2 shows the effects of divalent metal ions on β-galactosidase activity from E. coli and Pseudoalteromonas haloplanktis LMG P-1 9143

FIG. 3 shows effect of β-mercaptoethanol on kcat of β-galactosidase from E. coli and Pseudoalteromonas haloplanktis LMG P-19143 with ONPG as a substrate.

FIG. 4 shows the specific activities of β-galactosidase from E. coli and Pseudoalteromonas haloplanktisLMG P-19143, between 8 and 60° C., using ONPG as a substrate.

FIG. 5 shows the thermal stability of the activity of β-galacosidase from E. coli and from Pseudoalteromonas haloplanktis LMG P-19143 at 45° C., using ONPG as a substrate.

FIG. 6 shows the thermo-dependence of the physiological efficiency (kcat/km) of β-galactosidase of Pseudoalteromonas haloplanktis LMG P-19143, using ONPG as a substrate.

### DESCRIPTION OF THE PREFERRED EMBODIMENTS

Screening of a Bacterial Strain and Culture Conditions.

A bacterial strain was isolated and selected from sea water on necrosed algae at the J.S. Dumont d'Urville Antarctic Station (60°40′S; 40°01′E) The strain was identified as a Pseudoalteromonas haloplanktis by identification systems such the quantitative analysis of cellular fatty acid composition performed using a gas-liquid chromatography procedure known in the Art (Mergaert et al, 1993, Int. J. Syst. Bacteriol., 43, 162-173), using the Microbial Identification System (MIS, Microbial ID Inc., Newark, Del., U.S.A.). The peak recognition program was the MIS TSBA40 database for fatty acids and the chromatographic profiles were identified by comparison to the MIS TSBA database for aerobic bacteria (version 4.0).

The identified strain was then deposited according to the Budapest Treaty at the BCCM™ (Belgian Coordinated Collections of Microorganisms) with the following Accession Number: LMG P-19143, on the 4^{th} of Nov., 1999.

The screening of strains collected in the Antarctic, for showing a β-galactosidase activity, was carried out on L-agar plates containing 10 g/l bactotryptone, 5 g/l yeast extract, 25 g/l sea salts, 17 g/l agar (Difco) with 0,2% lactose, 32 mg/l X-Gal (5-Bromo-4-chloro-3indolyl-β-D-galactopyranoside) (Eurogentec) with or without 1 mM IPTG (isopropyl-thio-β-D-galactopyranoside) (Sigma); Growth properties were studied in L-Broth (10 g tryptone, 5 g yeast extract, 30 g sea salts in 1 L at pH8.5) containing 1% or 2% lactose. Cultures inoculated with 10 ml of a pre-culture grown at 4° C. were run at 250 rpm in 500 ml Erlenmeyer flasks containing 300 ml culture medium. After 115 hours culture, the absorbance of the culture was measured at 550 nm and the cells were pelleted and sonicated.

The definition of enzyme activity units can be defined according to the substrate used : with lactose as a substrate, the unit of activity is defined as the amount of enzyme which releases one micro-mole glucose in one minute under standard reaction conditions (temperature, pH). Another commonly used substrate is orthonitrophenyl-β-D galactopyranoside (ONPG) and in this case, the unit of activity is defined as the amount of enzyme which hydrolyses one micro-mole of ONPG in one minute under standard reaction conditions.

The degree of hydrolysis, defined as the percentage of lactose molecules cleaved, is most simply measured by determination of the amount of glucose released, or by changes in the physical properties of the hydrolysed lactose solution. Solution properties such as freezing point depression change as the disaccharide lactose is converted into the lower molecular weight monosaccharides glucose and galactose.

The intracellular β-galactosidase activity was assayed using ONPG as substrate. When 1 mM IPTG was added to the culture, the β-galactosidase activity was enhanced at least 2 times in the strain selected among the bacterial samples collected. This selected strain was a Gram negative and protease positive bacterium, chosen for its high β-galactosidase activity and its growth properties in liquid medium.

The strain was characterised and different growth conditions were tested. Sea salts at different concentrations were added to the culture medium: 5, 10, and 30 g/l with a lactose concentration of 10 or 20 g/l. The optimum growth medium was a rich medium comprising 2% lactose and 3% sea salts. In particular, the addition of sea salts to the growth medium enhanced the growth of the strain by a factor of ten.

The effects of adding IPTG in the growth medium were also studied and three IPTG concentrations were tested: 0.1 mM, 1 mM and 10 mM. It was observed that the addition of 1 mM IPTG to the growth medium after 44 hours of culture doubles the β-galactosidase activity in the cells.

FIG. 1a shows the growth rates of the strain at four temperatures: 4° C., 12° C., 18° C. and 25° C. by measuring the absorbance of the culture at 550 nm. The results obtained showed that temperatures above 4° C. induced faster growth rates but in the same time, reduced strain development. It is worth mentioning that growth rates are inaccurate as a sole criterion to determine the optimal growth temperature. This is clearly illustrated by the FIG. 1b showing the cell viability and the β-galactosidase activity of the strain of the invention at different temperatures.

The β-galactosidase from Escherichia coli used as a control was from Sigma (G2513).

The assay of β-galactosidase was carried out using 3 mM ONPG (ortho-nitrophenyl-β-galactopyranoside) as a chromogenic substrate in 100 mM sodium phosphate buffer, pH 7,3, 1 mM MgCl₂, 100 mM 2-mercaptoethanol (Sigma). Activities toward the chromogenic substrate were recorded in a thermostated Uvicon 860 Spectrophotometer (Kontron) at 25° C. and calculated on the basis of an extinction coefficient for o-nitrophenol of 3.5 mM⁻¹ cm⁻¹ at 410 nm (Miller, J. H. and Reznikoff, W. S., Eds. 1978; The Operon. Cold Spring Harbor Laboratory Press, NY.). Assays using lactose as a substrate were carried out using various concentrations of lactose. The reaction was stopped by boiling the sample in a water bath for 3 minutes. The galactose dehydrogenase assay was used to measure the amount of galactose released by the enzyme (Schachter H. 1975, Enzymatic microassays for D-Mannose, D-Glucose, D-Galactose, L-Fucose, and D-Glucosamine. Methods Enzymol., 41:3-10.) The specific activity of β-galactosidase is defined as micro-moles of galactose released per minute per mg of protein.

Purification and Characterization of a Cold-Active β-galactosidase from the Strain LMG P-19143 of the Present Invention

The Antarctic strain was cultivated at 4° C. for 5 days in ten litres of LB broth containing 2% lactose. After 44 hours, the culture was induced by ITPG (isopropyl-L-thio-β-D-galactopyranoside) to a final concentration of 1 mM and left for 68 hours.

The cells were harvested by centrifugation at 12,000× g for 60 minutes at 4° C. and re-suspended in 200 ml 50 mM MOPS (3-morpholinopropanesulfonic acid) buffer, pH 7.5. The cell-free extract was prepared by cell desintegration using the disruptor (LH-SGI Inceltech). 1 mM PMSF (Phenyl-methyl-sulphonyl-fluoride) was added to the crude extract to neutralise serine active proteases and debris were removed by centrifugation at 15,000×g for 30 minutes. Supernatant was then treated for two hours by protamine sulphate at a final concentration of 1 g/l to remove nucleic acids. After centrifugation for 30 minutes at 27000×g, the supernatant wad dialysed against 2×2 litres of MOPS buffer and then loaded on a DEAE-agarose column (35 ×2.5 cm) equilibrated in MOPS buffer and eluted with a NaCl linear gradient (500 ml-500 ml, 1 M, NaCl). Fractions containing β-galactosidase activity were pooled, concentrated up to 20 ml and dia-filtrated against MOPS buffer using a Minitan tangential flow ultra-filtration unit (Millipore) fitted with PTHK membrane (100 kDa molecular mass limit). The sample was then added to an affinity matrix of agarose derivatized with p-aminobenzyl-1-thio-β-D-galactopyranoside (Sigma A0414) (Steers E., Jr., Cuatrecasas P., and Pollard H., B., 1971, J. Biol. Chem. 246:196-200). The matrix containing the sample was washed with 1 M KCl and eluted with 100 mM lactose in MOPS buffer containing 1M KCl. The active fractions were pooled and applied on a Sephacryl S-300 column (95×3 cm) eluted with MOPS buffer.

Several steps were necessary to purify to homogeneity β-galactosidase from LMG P-19143. These steps are summarized in Table 1.

TABLE 1
Purification of the intracellular β-galactosidase from LMG P-19143
One unit of β-galactosidase is defined as the amount of the enzyme
required to release 1 μmole of nitrophenol min at pH 7.3 and at 20° C.

| | Vol | Protein | Total activity | Sp act | Recovery | Purification |
|---|---|---|---|---|---|---|
| Purification step | (ml) | (mg) | (μmol/min) | μmol/min/mg | (%) | (fold) |
| Crude extract | 200 | 1480 | 6954 | 4.7 | 100 | 1 |
| DEAE-agarose | 80 | 178.4 | 3101 | 1704 | 45 | 3.7 |
| Affinity Chromatography | 6 | 9.1 | 2509 | 276.5 | 36 | 58.8 |

Upon loading on DEAE sepharose column, β-galactosidase was eluted as a single peak at a NaCl concentration of approximately 400 nM. Although the affinity column decreased the yield of active β-galactosidase, it increased the purity by removing other remaining contaminant proteins. From 2 L culture grown under the conditions described above, the yield of purified β-galactosidase amounted to 10 ng. Following this procedure, the enzyme is 99% pure as determined by SDS-PAGE and has an estimated apparent molecular mass of 118 kDa. Ultrafiltration tests showed that β-galactosidase from LMG P-19143 is concentrated by an ultra-filtration membrane displaying a cut off of 300 kDa.

Analytical Procedures

Protein concentrations were determined by the method of Bradford (Bradford, M. M., 1976, Anal. Biochem. 72:248-254) using reagents from Pierce and bovine serum albumin as standard. For the purified enzyme, the following extinction coefficients at 280 nm were used : β-galactosidase from E. coli; 241590 M⁻¹ cm⁻¹, β-galactosidase from LMG P-1 9143; 195000 M⁻¹ cm⁻¹.

The NH₂-terminal amino acid sequence of the LMG P-19143 β-galactosidase was determined using a pulsed liquid phase protein sequencer (Procise Applied Biosystems 492).

SDS-polyacrylamide gel electrophoresis and isoelectric focusing were run essentially as described by the supplier of the electrophoresis equipment (Hoeffer Scientific Instruments). Isoelectric experiments were carried out using pH ranges 3.5-10 and 4-6 in 6% polyacrylamide gels containing 5.5% ampholytes. The anolyte was 0.02 M acetic acid and the catholyte was 0.02M NaOH.

The activation energy (Eₐ) was determined from the slope (−Eₐ/R) of Arrhenius plot and the thermodynamic activation parameters of the reaction were calculated according to the following equations:
ΔG*−ΔH*−TΔS* (Eq. 1)
ΔH*=Eₐ−RT (Eq. 2)
ΔS*=2.303R(log k_{cat}−10.753−logT+Eₐ2.303RT) (Eq. 3)

The isoelectric point of the β-galactosidase from LMG P-19143 was determined at 7.8; this value is higher than that of E. coli β-galactosidase which was found to be 4.6 [Wallenfels K. and Weil R., 1972, In “The enzymes” (Boyer, P. D., ed) Academic Press, New York 7:617-663].

To determine the optimal pH, the enzyme activity was measured in Michaelis's barbital sodium acetate buffer with pH values from 3 to 9.5 and Sorensen's glycin II buffer with pH values from 8.5 to 13. The pH optimum for the LMG P-19143 β-galactosidase activity was found to be at pH 8.5 which is slightly higher than that of E. coli enzyme. Over a pH range from 6.5 to 10, both mesophilic and psychrophilic enzymes retain 90% activity after 90 minutes and 60% after 24 hours exposure. The pH stability was optimum at pH 9. The stability of LMG P-19143 β-galactosidase was also tested in various buffers 50 mM MOPS, MES, TRIS and CHES at different pH values (from 5.5 to 9.7) for 20 hours. The enzyme stability is better in MOPS buffer at pH 7.5 and in MES buffer at pH 7.

The effect of various cations such as Zn²⁺, Mn²⁺, Cu²⁺, Ni²⁺, Li²⁺, Co²⁺, Ca²⁺, Na²⁺ and Fe²⁺ on the enzyme stability was also investigated. The activity of the enzyme was measured at time zero and then after 1, 2, 4 and 29 hours incubation at 4° C. The enzyme is stable in the presence of 0.1 to 1 mM Mg²⁺ and also in 0.1 mM Li²⁺ and 0.1 mM Ca²⁺. The LMG P-1 9143 β-galactosidase is inhibited by Cu²⁺, Ni²⁺ and Zn²⁺ at concentrations from 0.1 to 10 mM and by 10 mM Fe²⁺.

To determine the effect of divalent metal ions on activity, assays were performed in 100 mM phosphate buffer at 25° C. and pH 7.5. The enzyme preparation was treated with 5 mM EDTA to complex metal ions. After this treatment, the enzyme showed less than 10% of its initial activity. Addition of 10 mM magnesium restored and enhanced two times the activity of LMG P-19143 β-galactosidase, 20 mM calcium or 5 mM manganese restored partially the activity of the enzyme. Addition of 20 mM Mg²⁺ restored the activity of E. coli β-galactosidase, 20 mM Ca²⁺ or Mn²⁺5 mM restored partially the activity of E. coli β-galactosidase as shown in FIG. 2.

The effect of K+ was determined by assaying activity in 100 mM phosphate buffer containing KCl at concentrations from 0 to 100 mM. LMG P-19143 β-galactosidase optimal activity was recorded using a KCl concentration of 80 mM whereas the E. coli β-galactosidase optimal activity was recorded at a KCl concentration of 40 mM. At theses concentrations, KCl stimulated the activity of both enzymes by a factor of 1.5.

FIG. 3 shows the effect of 2-mercaptoethanol on β-galactosidase activity evaluated in the same conditions. Optimal activity of the LMG P-19143 enzyme was recorded at 80 mM 2-mercaptoethanol and that of E. coli enzyme at 40 mM 2-mercaptoethanol. At these concentrations, the reducing agent stimulated LMG P-19143 enzyme activity twofold and E. coli enzyme by a factor of 1.5.

FIG. 4 shows the effect of temperature on the β-galactosidase activity determined by assaying the enzyme at various temperatures from 5° C. to 60° C. using ONPG as a substrate. The thermo-dependency of the activity of LMG P-19143 β-galactosidase shows a shift of the apparent optimal temperature of activity by 10° C. toward low temperatures when compared to the E. coli enzyme. At 8° C., the kcat (s⁻¹) of the LMG P-19143 enzyme is twice as high as that of E. coli enzyme. Theses curves have been used to construct Arrhenius plots and to calculate the activation energy parameters of the reaction as shown in table 2.

TABLE 2
Kinetic and thermodynamic activation parameters of β-galactosidase
activity at 20° C. using ONPG as substrate

| Parameter | LMG P-19143 | E. coli |
|---|---|---|
| k_{cat} (s⁻¹) | 408 | 199 |
| Eₐ (kJ mol⁻¹)^{a} | 15.5 | 36.2 |
| ΔG* (kJ mol⁻¹) | 60.5 | 62.4 |
| ΔH* (kJ mol⁻¹) | 13.1 | 33.8 |
| ΔS* (J mol⁻¹ K⁻¹) | −162 | −97.6 |

The lower free energy of activation (ΔG*) of LMG P-19143 β-galactosidase correlates well with its higher specific activity, but the contribution of the enthalpy term (ΔH*) and of the entropy (TΔS*) to ΔG* also differs in both enzymes.

Thermal stability was determined by incubating the enzymes at different temperatures and periodically withdrawing for assay at 25° C. FIG. 5 shows that, at 45° C., the half-life of the LMG P-19143 β-galactosidase (30 min.) is 12 times lower than the half-life (6 hours) of the E. coli enzyme.

Assays were performed with ONPG as a substrate at various concentrations and at different temperatures to determine Km and Vₘₐₓ values. At 10° C., the apparent Km is nearly the same for the two enzymes. Moreover, as shown in FIG. 6, the physiological efficiency (kcat/km) is about three times higher for the LMG P-19143 β-galactosidase.

Km was also determined at 25° C. with lactose (1 mM to 50 mM) as a substrate. Apparent Km was 2,4 mM with the LMG P-19143 enzyme and 13 mM with E. coli enzyme. LMG P-19143 β-galactosidase displays a kcat of 34.1 U/mg and the E. coli enzyme a kcat of only 2.15 U/mg. The physiological efficiency (kcat/km) of the cold-adapted enzyme is ninety times higher than that of the E. coli β-galactosidase.

The β-galactosidase of the present invention being purified and its physiological properties being established, a further step was to investigate the genetic characteristics of it.

DNA Isolation

DNA from strain LMG P-19143 was isolated by a modification of the method of Brahamsha, (Brahamsha B., and E. P. Greenberg, 1987, J. Bacteriol. 169:3764-3769). The lysozyme concentration was increased to 1 mg/ml and the cells were treated for 30 minutes at 37° C. The extract was then incubated in 0.5% sodium dodecyl sulphate (SDS) and proteinase K (1 μg/ml final) at 55° C. for one hour. The resulting lysate was then extracted three times with an equal volume of phenol/chloroform (50% V/V) followed by a chloroform extraction. The DNA was then precipitated with ethanol and suspended in TE buffer (10 mM Tris. Cl, 1 mM EDTA, pH 8).

Cloning

The restriction and ligation enzymes were supplied by Gibco and BMI. Genomic DNA of LMG P-19143 extracted according to the protocol described above, was digested with Sau 3AI, Hind III, Pst I or Sph I and the resulting fragments were inserted into the corresponding sites of the plasmid pSP 73 (Promega). The ligated DNA was transformed in E. coli dH5^{s}ₓ whose endogeneous β-galactosidase is inactive by mutation. Indeed, the plasmid pSP 73 lacks a portion of the lac Z gene which provides essential ^{s}ₓ-complementation for endogeneous β-galactosidase of E. coli dH5^{s}ₓ. The plasmid pSP 73 is directly derived from pBR 322 (Promega, USA). It displays an oligonucleotides sequence of 2464 pb (GenBank: EMBL accession number X65333). The transformants were selected on L-agar plates containing 50 βg ampicillin/ml, 0.01% X-Gal (5-bromo-4-chloro-3-indolyl-^{s}ₓ-D-galactopyranoside) and 100 μM IPTG. After two days incubation at 25° C., the (β-galactosidase-positive colonies became blue. The β-galactosidase gene-containing DNA fragment was subcloned into the polylinker of pSP 73 by digestion with Xba I, Bgl II or Eco RI and plasmid self ligation. The sub-clones were analyzed by testing β-galactosidase activity on L-agar plates containing 50 βg ampicillin/ml, 0.01% X-gal and 100 βM IPTG.

For DNA sequencing, the sub-clone Eco RI was ligated in pK 19 (Pridmore R. D., 1987, Gene 56:309-312). DNA sequencing was performed using the chromosome walking technique with 5′ Fluorescein labeled primers. The products of the sequencing reaction were analyzed on ALF DNA sequencer (Pharmacia, Sweden). Synthetic oligonucleotides used as primers were from Eurogentec S. A.

The N-terminal amino sequence of the purified enzyme according to the present invention has been determined and alignment of the first nineteen amino acids of LMG P-19143β-galactosidase with the N-terminal sequence of the E. coli enzyme showed ten conserved positions.

Cloning of the LMG P-19143β-galactosidase Gene

Four genomic libraries of LMG P-19143 DNA were constructed by restriction digestion of DNA with Sau 3AI, Hind III, Pst I or Sph I and ligation into the corresponding sites of the vector pSP 73.

pSP 73 plasmid lacks the lac Z^{s}ₓ fragment which could complement the E. coli dH5^{s}ₓ deleted β-galactosidase. Transformants of E. coli dH5^{s}ₓ containing the pSP 73 vector without any insert produced white colonies on X-gal plates. From the colonies screened at 25° C., three β-galactosidase positive colonies were obtained. The DNA inserts of these three β-galactosidase positive transformants were the same and this insert is a fragment Pst I-Pst I of nearly 9 kb.

A restriction map of clone Pst I-Pst I was generated and fragments were subcloned to determine the smallest fragment which could encode the β-galactosidase gene. The colonies obtained were analyzed on the basis of β-galactosidase activity on X-gal plates. Three clones which produced β-galactosidase activity were found; theses clones were the result of restriction digestion Xba I, Bgl II and Eco RI. The Eco RI fragment was chosen for sequencing.

Nucleotide Sequence of the LMG P-19143 β-galactosidase Gene

The Eco RI-Pst I fragment of 5088 bp has been totally sequenced. A single large open reading frame was found starting with an ATG at nucleotide 1531 and ending with a TAG at nucleotide 4649; it has been sequenced four times on both strands and its sequence is shown in SEQ ID NO: 1. The first NH₂ terminal amino acids of the native protein determined by EDMAN degradation could be recognized following the ATG of the open reading frame. Therefore the protein corresponds to 1038 amino acids with a calculated Mᵣ of 118068. The predicted amino acid sequence of the sequenced gene was compared with protein sequences databases with “BLAST network service” program. The protein sequence shown in SEQ ID NO: 2 was aligned with E. coli lac Z gene by “TFASTA” program with 51% sequence similarities. The LMG P-19143 gene was so designated lac Z on the basis of its sequence similarities with the lac Z from E. coli. The alignment showed that the proposed active-site residues in E. coli lac Z; Glu-461, Glu-537, Met 502 and Tyr 503 are conserved in the LMG P-19143 sequence. The alignment with other lac Z β-galactosidase showed significant homology surrounding Glu-461 and Glu-537, forming the consensus sequences. The tyrosine residue which is important for the reaction is also conserved in the LMG P-19143 sequence.

The β-galactosidase protein sequence analysis had allowed to identify structural features typical of cold-adapted enzymes. For the LMG P-19143 protein, Arginine content (39) and Arg/Arg+Lys ratio (0.47) are smaller than for E. coli β-galactosidase, 66 and 0.77, respectively. The proline residues content is also smaller for the cold-adapted enzyme (46 and 62 respectively) and its glycine content was higher within the 15 amino acids around the catalytic residue Glu 461.

Alignment with E. coli lac Z gene showed three insertions in the LMP-19143 lac Z gene. These insertions of 4, 5 and 9 residues are located at Glu 78, Gln 634 and Asn 739 respectively.

The LMG P-19143 β-galactosidase shares structural properties with the mesophilic E. coli β-galactosidase. The apparent sub-unit mass of the LMG P-19143 β-galactosidase is comparable to that of E. coli enzyme. The cold-adapted enzyme is a multimer since it is concentrated by an ultra-filtration membrane of 300 kDa cut off. The sub-unit is long of 1038 amino acids with a Mr of 118,068, which is slightly higher than that of E. coli lac Z enzyme with 1,023 amino acids.

The β-galactosidase from LMG P-19143 shows an optimal pH value of 8.5 for both stability and activity which is comparable to what is observed for the E. coli β-galactosidase. The two enzymes have a good activity within the pH range of 6.6-10, this would allow the efficient treatment of milk, the pH of which is 6.6.

LMG P-19143 and E. coli β-galactosidase are activated by 2-mercaptoethanol. SH-groups may be involved in the catalytic process but other data show that certain SH-groups may be important for maintaining the active conformation of the enzyme [Wallenfels K. and Weil R., 1972. In “The enzymes” (Boyer, P. D., ed.) Academic Press, New York 7:617-663].

As many fungal and bacterial β-galactosidase, LMG P-19143 and E. coli enzymes require divalent cations for activity. Indeed, addition of EDTA, a chelating agent, to the assay mixture leads to enzyme inactivation. Addition of magnesium, calcium or manganese restored the activity. So LMG P-19143 β-galactosidase is a metallo-enzyme having a strict requirement for divalent metal ions as suggested for E. coli β-galactosidase by Wallenfels K. and Weil R. in “The enzymes” (Boyer, P. D., ed., Academic Press, New York 7:617-663, 1972). Moreover the three-dimensional structure of E. coli β-galactosidase showed two bound magnesium per monomer (Jacobson R. H., Zhang X-J., DuBose R. F. and Matthews B. W., 1994. Nature 369:761-766).

The alignment of LMG P-19143 sequence with other lac Z β-galactosidase showed the conservation of the amino acid residues involved in catalysis. The proposed mechanism of action for the E. coli lac Z β-galactosidase involves a double displacement reaction in which the enzyme forms and hydrolyses a glycosyl-enzyme intermediate via oxocarbonium ion-like transition states (Gebler, J. C., R. Aebersold, and S. G. Withers, 1992. J. Biol. Chem. 267:11126-11130). These authors identified Glu-537 as the nucleophilic amino acid and suggested that Glu-461 serves as the general acid/base catalyst which protonates the galactosyl transition state intermediate and deprotonates the attaching water in the E. coli lac Z protein. The analysis of the three-dimensional structure of β-galactosidase from E. coli showed that residues Glu 461, Met 502, Tyr 503 and Glu 537 are found closed together and formed a pocket that was identified as the substrate binding site. Glu 537 is situated on the opposite site of the cavity and oriented through hydrogen bonding with Tyr 503 and Arg 388 (Jacobson R. H., Zhang X-J., DuBose R. F. and Matthews B. W., 1994. Nature 369:761-766). These residues are also conserved in the LMG P-19143 sequence. Affinity labeling of β-galactosidase has identified Met-502 as a non-essential active site residue, whereas the suggestion that the adjacent residue, Tyr-503, may play a direct role as an acid/base catalyst, was supported by subsequent analysis of mutants modified at this position (Gebler, J. C., R. Aebersold, and S. G. Withers, 1992. J. Biol. Chem. 267:11126-11130). Among homologous β-galactosidase sequences, residues that form the active-site pocket are highly conserved (Jacobson R. H., Zhang X-J., DuBose R. F. and Matthews B. W., 1994. Nature 369:761-766).

LMG P-19143 β-galactosidase also shares common properties with cold-adapted enzymes (Feller G., Arpigny J. L., Narinx E., and Gerday C., 1997. Comp. Biochem. Physiol. 118A:495-499).

Indeed the cold β-galactosidase displays a lower apparent optimum temperature of activity and a lower thermal stability than the E. coli enzyme. Moreover over the temperature range of 0-40° C., the lever of turnover (kcat) of LMG P-19143 β-galactosidase towards ONPG is higher than that E. coli enzyme. This difference in favour of the cold-adapted enzyme is dramatically increased when lactose is used as substrate (fifteen times at 25° C.). The thermodynamic parameters showed in (Table 2) are consistent with the fact that the activated state of the complex is reached through a minimum of entropy change and with a lower activation enthalpy when compared to E. coli β-galactosidase.

With ONPG as a substrate, the km values are, at low temperature, comparable for both enzymes. However, since kcat value is significantly higher to LMG P-19143 β-galactosidase, the physiological efficiency is also higher for the LMG P-19143 β-galactosidase.

With lactose as a substrate at 25° C., km is five times lower for the LMG P-19143 enzyme and the physiological efficiency (kcat/Km) is therefore eighty times as high as that of E. coli β-galactosidase.

The above mentioned data allow to clarify to some extent some questions raised about the possible differences in the molecular adaptation of intracellular enzymes when compared to extracellular ones (Gerday et al., 1998). Indeed in a few cases: citrate synthase and β-galactosidase, the specific activity was not higher than the mesophilic counterparts whereas thermostability was, in all cases, much lower than that of mesophilic enzymes.

The alignment of the amino acid sequence of LMG P-19143 β-galactosidase with that of E. coli β-galactosidase shows three insertions of 4, 5 and 9 residues. If located in surface loops, these insertions could contribute to increase the plasticity of the molecular edifice as also suggested in the case of subtilisin S41 (Davail S., Feller G., Narinx E. and Gerday C., 1994, J. Biol. Chem. 269:17448-17453). Nevertheless the involvement of indels in cold-adaptation is strongly specific to each enzyme type and can not be generalized (Feller G., Arpigny J. L., Narinx E., and Gerday C., 1997. Comp. Biochem. Physiol. 118A:495-499).

As in the case of several cold-adapted enzymes, the LMG P-19143 β-galactosidase arginine content (55) is lower than that of its mesophilic counterpart (66). Arginine residues play a significant role in thermal adaptation. Indeed, the charge resonance of the guanidium group gives arginine the possibility to form more than one salt bridge (Mrabet et al., 1992) as well as multiple hydrogen bonds with surrounding acceptors (Borders, CL. L, Broadwater, J. A.; Bekeny, P. A., Salmon, J. A., Lee, A. S., Eldrige, A. M., Pett, V. B., 1994, Protein Sci. 3:541-548). The multivalent character of arginine certainly account for its low occurrence in many cold-adapted enzymes and in enzymes of low stability in general (Menendez-Ariaz, M. and Argos, P, 1989, J. Mol. Biol. 206:397-406).

The cold-adapted enzyme also shows a lower content of proline (46 compared to 62 in the mesophilic enzyme). The cyclic structure of proline severely impairs the rotations about its N—C^{s}^{ₓ} bond. So, the presence of this residue in a protein greatly reduces the number of possible local conformations of the molecular backbone. This reduces the conformational entropy of the unfolded state and confers more rigidity to the native protein (Matthews et al., 1987).

On the contrary, LMG P-19143 β-galactosidase glycine content is lower than that E. coli enzyme. Glycine, which has a side chain, increases the degrees of freedom of the unfolded polypeptide backbone. The replacement of one Gly by another residue in theory can reduce the backbone flexibility and destabilizes the unfolded state by as much as 3.3 kJ mol⁻¹ at 65° C. (Nemethy et al., 1966). Nevertheless it has been suggested that the stacking of Gly around the catalytic residues provides high active site flexibility (Karplus and Shultz, 1985).

To conclude, LMG P-19143 β-galactosidase is a cold-adapted enzyme that is much more active at low and moderate temperatures when compared to the mesophilic enzyme from E. coli. Moreover the ideal optimum pH range (6-8) is suitable for lactose hydrolysis in milk and dairy products.

1. An isolated psychrophilic bacterium Pseudoalteromonas haloplanktis, or a variant or mutant thereof that produces a cold-active beta galactosidase that is specific for lactose and has stable enzymatic activity at a temperature below 8° C.

2. A method for producing a cold-active beta galactosidase that is specific for lactose and has stable enzymatic activity at a temperature below 8° C., comprising
culturing an isolated psychrophilic bacterium Pseudoalteromonas haloplanktis, or a variant or mutant thereof that produces a cold-active beta galactosidase that is specific for lactose and has stable enzymatic activity at a temperature below 8° C., under conditions effective for producing the beta galactosidase,
and harvesting the beta galactosidase from the bacterium.

3. An isolated DNA comprising a sequence which encodes a cold-active beta galatosidase that is specific for lactose and has a stable enzymatic activity at a temperature below 8° C.

4. The isolated DNA of claim 3, which is isolated from a beta galactosidase-producing microorganism.

5. The isolated DNA of claim 3, which is isolated from the psychrophilic bacterium Pseudoalteromonas haloplanktis.

6. The isolated DNA of claim 5, wherein the psychrophilic bacterium Pseudoalteromonas haloplanklis has the BCCM™ Accession Number LMG P-19143.

7. The isolated DNA of claim 3, which comprises the sequence shown in SEQ ID NO: 1.

8. The isolated DNA of claim 3, which encodes a polypeptide comprising the sequence shown in SEQ ID NO: 2.

9. An isolated DNA which hybridizes to a DNA that encodes a cold-active beta galactosidase that is specific for lactose and has a stable enzymatic activity at a temperature below 8° C.

10. An isolated DNA which hybridizes to a DNA that encodes a cold-active beta galatosidase that is specific for lactose, has a stable enzymatic activity at a temperature below 8° C., and is produced by the psychrophilic bacterium Pseudoalteromonas haloplanktis.

11. A recombinant plasmid comprising the DNA of claim 3, which expresses the cold-active beta galactosidase.

12. A recombinant plasmid comprising the DNA of claim 5, which expresses the cold-active beta galatosidase.

13. A recombinant plasmid comprising the DNA sequence of claim 3, operatively linked to an expression control sequence.

14. A recombinant plasmid comprising the DNA sequence of claim 5, operatively linked to an expression control sequence.

15. A cell transformed with a recombinant plasmid of claim 11.

16. A cell transformed with a recombinant plasmid of claim 12.

17. A cell transformed with a recombinant plasmid of claim 13.

18. A cell transformed with a recombinant plasmid of claim 14.

19. The cell of claim 15, which is a bacterium or a yeast cell.

20. The cell of claim 16, which is a bacterium or a yeast cell.

21. The cell of claim 17, which is a bacterium or a yeast cell.

22. The cell of claim 18, which is a bacterium or a yeast cell.


SEQUENCE LISTING (2 sequences)

SEQ ID NO: 1 - DNA, 3171 bp, Pseudoalteromonas haloplanktis
tagctatatttagcgccattataattgcccgtttatgcaacaggaataaacatgacctctttacagcacataattaatcgtcgcgattgggaaaatccaattacagtacaagttaatcaagtaaaagcacatagcccacttaacggctttaaaacaattgaagacgcccgtgaaaatacacagtcgcagaagaaaagtttaaacgggcagtgggattttaaattatttgataagcccgaagcggtcgatgagtcgttattgtatgagaagataagtaaagagctaagcggcgactggcaaagtattactgtgccttctaactggcaactacacggctttgataaacccatttactgtaatgttaaatacccatttgcagtaaacccgccatttgtaccaagcgataaccctactggttgttaccgcactgaatttacaatcacacctgagcagttaacgcagcgtaaccatataatttttgaaggcgttaactcggcttttcatctttggtgtaacgggcagtgggtggggtattcacaagatagccgcttaccgagcgaatttgatttaagtgagcttttagttgtcggtactaaccgtattgccgttatggttattcgttggagtgatggcagttatttagaagatcaggatatgtggtggctaagcggtatttttcgcgatgttaacttacttacaaaaccgcaaagccaaatacgcgatgtgtttataacccccgatttagacgcttgctatcgcgatgcaacgctacatataaaaactgcgataaatgcgccaaataactaccaagtagcagtacagatttttgatggtaaaacatcactgtgcgagccgaaaattcaaagcactaacaataaacgtgttgatgaaaaaggggggtggagcgatgtcgtatttcaaacaatagcaatacgaagccctaaaaagtggaccgccgaaacgccgtacttatatcgttgcgtagtaagcctgcttgatgaacaaggcaatacagtcgacgttgaagcctataacattggttttagaaaagtagaaatgcttaacgggcagctgtgtgtaaatggcaaaccgttacttatacggggtgttaaccgacacgaacatcacccagaaaacggccatgctgttagcactgccgatatgattgaagatattaagctgatgaagcaaaataactttaatgccgtacgtacagctcattaccctaaccatccacttttttacgagctatgtgacgagctaggtttatacgtggttgatgaagcgaatatagaaacccatggcatgtttcctatggggcgtttagcaagcgatccgctatgggcaggtgcatttatgtcgcgttatacgcaaatggttgagcgcgataaaaaccacgcctcaattattatttggtcacttggaaacgaatgcgggcacggcgcaaatcatgatgctatgtatggctggtcaaaaagctttgacccttctcgcccagtgcaatacgagggcggcggtgcaaacacgacagctaccgatattatttgcccaatgtactcccgtgtagataccgatattaaagacgatgcggtacctaagtattcaattaaaaaatggctgagcttaccgggtgaaactcgtccacttattttatgtgagtacgcccatgctatgggtaatagcttaggtagctttgacgattactggcaggcatttagagaatacccacggctgcaaggcggctttatttgggattgggtagatcaaggtttatctaaaattgacgagaacggcaagcattattgggcttacggcggcgactttggtgatgaactaaacgaccgccagttttgtataaacggcttattgttcccggatcgtacaccgcatcctagcctatttgaagctaaatacagccagcaacatttacaatttacactgcgcgagcaaaatcaaaatcaaaaccaaaaccaatacagcattgatgtatttagcgattacgtatttaggcacaccgataacgaaaaactcgtttggcaattaatacaaaatggcgtgtgtgttgagcaaggcgaaatggcacttaatattgctccgcaaagtacgcacactttaaccattaaaactaaaacagcgtttgagcatggtgcgcaatattaccttaatttagatgtagcactaattaacgactcacactttgcaaacgctaatcacgttatggattcagaacagtttaagcttataaatagtaataatttaaacagtaaatcatttgcatcagctacagagaaaagcgttataagtgttaatgaaaccgactcccacctaagtattgaaaacaatacatttaaacttgtttttaatcaacaatcaggacttatagagcagtggttacaagacgatacacaggttattagtagcccactggttgataacttttatcgtgccccacttgataacgacattggtgtaagcgaagtggacaacctagaccctaatgcatgggaagcacgctggtcgcgcgcaggtatagggcaatggcagcgcacatgtagctcaatcaatgctgtgcaatcaagcgttgatgtccgtattacttgtgtatttaattacgaatttaatggcgtgctacaagcacaaacacagtggctatatacgctcaataatacaggtactattagcttaaatgttgatgtgaacttaaacgacaccctaccaccaatgccgcgaatagggttaagtacaacgattaacaagcaaagcgatacaaaagtaaactggctagggttaggtccttttgaaaactacccagatcgtaaatccgctgcacgttttggttattacagcttgagcttaaatgagctatatacaccgtatatattcccaactgataacggtctgcgtagcgattgccaattactgagcattaataacttaatcgtgactggcgcgtttttgtttgccgccagtgagtattcgcaaaatatgctaacgcaagctaaacacactaacgaactaattgctgatgattgcattcatgtacatattgatcatcaacatatgggtgtaggtggcgatgattcgtggagtccaagtacccataaagagtatttattagagcaaaaaaattataattactcgcttacacttactggggggattacaacttaa

SEQ ID NO: 2 - protein, 1039 aa, Pseudoalteromonas haloplanktis
  ACT_SITE (460)
  ACT_SITE (501)
  ACT_SITE (502)
  ACT_SITE (536)
  SIMILAR (533)..(543)
  SIMILAR (455)..(460)
MTSLQHIINRRDWENPITVQVNQVKAHSPLNGFKTIEDARENTQSQKKSLNGQWDFKLFDKPEAVDESLLYEKISKELSGDWQSITVPSNWQLHGFDKPIYCNVKYPFAVNPPFVPSDNPTGCYRTEFTITPEQLTQRNHIIFEGVNSAFHLWCNGQWVGYSQDSRLPSEFDLSELLVVGTNRIAVMVIRWSDGSYLEDQDMWWLSGIFRDVNLLTKPQSQIRDVFITPDLDACYRDATLHIKTAINAPNNYQVAVQIFDGKTSLCEPKIQSTNNKRVDEKGGWSDVVFQTIAIRSPKKWTAETPYLYRCVVSLLDEQGNTVDVEAYNIGFRKVEMLNGQLCVNGKPLLIRGVNRHEHHPENGHAVSTADMIEDIKLMKQNNFNAVRTAHYPNHPLFYELCDELGLYVVDEANIETHGMFPMGRLASDPLWAGAFMSRYTQMVERDKNHASIIIWSLGNECGHGANHDAMYGWSKSFDPSRPVQYEGGGANTTATDIICPMYSRVDTDIKDDAVPKYSIKKWLSLPGETRPLILCEYAHAMGNSLGSFDDYWQAFREYPRLQGGFIWDWVDQGLSKIDENGKHYWAYGGDFGDELNDRQFCINGLLFPDRTPHPSLFEAKYSQQHLQFTLREQNQNQNQNQYSIDVFSDYVFRHTDNEKLVWQLIQNGVCVEQGEMALNIAPQSTHTLTIKTKTAFEHGAQYYLNLDVALINDSHFANANHVMDSEQFKLINSNNLNSKSFASATEKSVISVNETDSHLSIENNTFKLVFNQQSGLIEQWLQDDTQVISSPLVDNFYRAPLDNDIGVSEVDNLDPNAWEARWSRAGIGQWQRTCSSINAVQSSVDVRITCVFNYEFNGVLQAQTQWLYTLNNTGTISLNVDVNLNDTLPPMPRIGLSTTINKQSDTKVNWLGLGPFENYPDRKSAARFGYYSLSLNELYTPYIFPTDNGLRSDCQLLSINNLIVTGAFLFAASEYSQNMLTQAKHTNELIADDCIHVHIDHQHMGVGGDDSWSPSTHKEYLLEQKNYNYSLTLTGGITT